(19) United States
(12) Patent Application Publication
(10) Pub. No.: US 2008/0081936 A1
(43) Pub. Date: Apr. 3, 2008

(54) Integrated processing of methanol to olefins

(76) Inventors: Andrea G. Bozzano, Northbrook, IL (US); Bipin V. Vora, Naperville, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/540,802

(22) Filed: Sep. 29, 2006

(51) Int. Cl. C07C 1/00

(52) U.S. Cl. 585/639

(57) ABSTRACT

Processing schemes and arrangements for the production of olefins and, more particularly, for the production of light olefins from a methanol feedstock are provided. Such processing schemes and arrangements integrate oxygenate conversion at higher pressures and with subsequent heavy olefins conversion processing to produce additional light olefin products.

### BACKGROUND OF THE INVENTION

This invention relates generally to the conversion of oxygenates to olefins and, more particularly, to light olefins, via integrated processing.

A major portion of the worldwide petrochemical industry is involved with the production of light olefin materials and their subsequent use in the production of numerous important chemical products. Such production and use of light olefin materials may involve various well-known chemical reactions including, for example, polymerization, oligomerization, and alkylation reactions. Light olefins generally include ethylene, propylene and mixtures thereof. These light olefins are essential building blocks used in the modern petrochemical and chemical industries. A major source for light olefins in present day refining is the steam cracking of petroleum feeds. For various reasons including geographical, economic, political and diminished supply considerations, the art has long sought sources other than petroleum for the massive quantities of raw materials that are needed to supply the demand for these light olefin materials.

The search for alternative materials for light olefin production has led to the use of oxygenates such as alcohols and, more particularly, to the use of methanol, ethanol, and higher alcohols or their derivatives or other oxygenates such as dimethyl ether, diethyl ether, etc., for example. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins.

Such processing, wherein the oxygenate-containing feed is primarily methanol or a methanol-water combination (including crude methanol), typically results in the release of significant quantities of water upon the sought conversion of such feeds to light olefins. For example, such processing normally involves the release of about 2 mols of water per mol of ethylene formed and the release of about 3 mols of water per mol of propylene formed. The presence of such increased relative amounts of water can significantly increase the potential for hydrothermal damage to the oxygenate conversion catalyst. Moreover, the presence of such increased relative amounts of water significantly increases the volumetric flow rate of the reactor effluent, resulting in the need for larger sized vessels and associated processing and operating equipment.

U.S. Pat. No. 5,714,662 to Vora et al., the disclosure of which is hereby incorporated by reference in its entirety, discloses a process for the production of light olefins from a hydrocarbon gas stream by a combination of reforming, oxygenate production, and oxygenate conversion wherein a crude methanol stream (produced in the production of oxygenates and comprising methanol, light ends, and heavier alcohols) is passed directly to an oxygenate conversion zone for the production of light olefins.

While such processing has proven to be effective for olefin production, further improvements have been desired and sought. For example, there is an ongoing desire and need for reducing the size and consequently the cost of required reaction vessels. Further, there is an ongoing desire and need for processing schemes and arrangements that can more readily handle and manage either or both the heat of reaction and byproduct water associated with such processing. Still further, there is an ongoing desire and need for processing schemes and arrangements that produce or result in increased relative amounts of light olefins.

### SUMMARY OF THE INVENTION

A general object of the invention is to provide improved processing schemes and arrangements for the production of olefins, particularly light olefins.

A more specific objective of the invention is to overcome one or more of the problems described above.

The general object of the invention can be attained, at least in part, through specified methods for producing light olefins. In accordance with one embodiment, there is provided a method for producing light olefins that involves contacting a methanol-containing feedstock in a methanol conversion reactor zone with a catalyst and at reaction conditions effective to produce a methanol conversion reactor zone effluent comprising dimethyl ether and water. At least a portion of the water is removed from the methanol conversion reactor zone effluent to form a first process stream comprising dimethyl ether and having a reduced water content. A feed comprising at least a portion of the first process stream is contacted in an oxygenate conversion reactor zone with an oxygenate conversion catalyst at oxygenate conversion reaction conditions, including an oxygenate conversion reaction pressure of at least about 240 kPa absolute, effective to convert at least a portion of the feed to an oxygenate conversion product stream comprising light olefins and heavy olefins. At least a portion of the oxygenate conversion product stream heavy olefins are reacted in a heavy olefins conversion zone to form a heavy olefins conversion zone effluent stream comprising additional light olefins. At least a portion of the additional light olefins are subsequently recovered from the heavy olefins conversion zone effluent stream.

The prior art generally fails to processing schemes and arrangements for the production of olefins and, more particularly, for the production of light olefins from an oxygenate-containing feed and which processing schemes and arrangements are as simple, effective and/or efficient as may be desired. More particularly, the prior art generally fails to provide such processing schemes and arrangements that address issues such as relating to water co-production, light olefin production with desirably increased propylene to ethylene ratios and carbon efficiency for light olefin production as simply, effectively and/or efficiently as may be desired.

A method for producing light olefins, in accordance with another embodiment, involves contacting a methanol-containing feedstock in a methanol conversion reactor zone with a catalyst and at reaction conditions effective to produce a methanol conversion reactor zone effluent comprising dimethyl ether and water. At least a portion of the water is removed from the methanol conversion reactor zone effluent to form a first process stream comprising dimethyl ether and having a reduced water content. A feed comprising at least a portion of the first process stream can then be contacted in an oxygenate conversion reactor zone with an oxygenate conversion catalyst at oxygenate conversion reaction conditions effective to convert at least a portion of the feed to an oxygenate conversion product stream comprising light olefins and heavy olefins. The oxygenate conversion reaction conditions desirably include an oxygenate conversion reaction pressure in a range of at least 300 kPa absolute to 450 kPa absolute. At least a portion of the oxygenate conversion product stream heavy olefins can subsequently be reacted in a heavy olefins conversion zone via at least one of an olefin cracking reaction and a metathesis reaction to form a heavy olefins conversion zone effluent stream comprising additional light olefins. At least a portion of the additional light olefins can subsequently be recovered from the heavy olefins conversion zone effluent stream.

There is also provided a system for producing light olefins. In accordance with one preferred embodiment, such a system includes a methanol conversion reactor zone for contacting a methanol-containing feedstock with a catalyst and at reaction conditions effective to produce a methanol conversion reactor zone effluent comprising dimethyl ether and water. A first separator is provided. The first separator is effective to separate at least a portion of the water from the methanol conversion reactor zone effluent to form a first process stream comprising dimethyl ether and having a reduced water content. An oxygenate conversion reactor zone is provided for contacting a feed comprising at least a portion of the first process stream dimethyl ether with an oxygenate conversion with a catalyst and at reaction conditions including a reaction pressure of at least 240 kPa absolute effective to convert at least a portion of the feed to an oxygenate conversion product stream comprising light olefins and heavy olefins. The system also includes a heavy olefins conversion zone effective to convert oxygenate conversion product stream heavy olefins to form a heavy olefins conversion zone effluent stream comprising additional light olefins. The system further includes a recovery zone for recovering at least a portion of the additional light olefins from the heavy olefins conversion zone effluent stream.

As used herein, references to “light olefins” are to be understood to generally refer to C₂ and C₃ olefins, i.e., ethylene and propylene.

In the subject context, the term “heavy olefins” generally refers to C₄-C₆ olefins.

“Oxygenates” are hydrocarbons that contain one or more oxygen atoms. Typical oxygenates include alcohols and ethers, for example.

“Carbon oxide” refers to carbon dioxide and/or carbon monoxide.

References to “Cₓ hydrocarbon” are to be understood to refer to hydrocarbon molecules having the number of carbon atoms represented by the subscript “x”. Similarly, the term “Cₓ-containing stream” refers to a stream that contains Cₓ hydrocarbon. The term “Cₓ+hydrocarbons” refers to hydrocarbon molecules having the number of carbon atoms represented by the subscript “x” or greater. For example, “C₄+hydrocarbons” include C₄, C₅ and higher carbon number hydrocarbons. The term “Cₓ−hydrocarbons” refers to hydrocarbon molecules having the number of carbon atoms represented by the subscript “x” or less. For example, “C₄−hydrocarbons” include C₄, C₃ and lower carbon number hydrocarbons.

“RWD” column or zone refers to a Reaction With Distillation column or zone such as can generally serve to combine reaction and distillation processing in a single processing apparatus.

Other objects and advantages will be apparent to those skilled in the art from the following detailed description taken in conjunction with the appended claims and drawings.

### BRIEF DESCRIPTION OF THE DRAWINGS

FIG. 1 is a simplified schematic diagram of an integrated system for the processing of an oxygenate-containing feedstock to olefins, particularly light olefins, in accordance with one embodiment.

FIG. 2 is a simplified schematic diagram of an integrated system for the processing of an oxygenate-containing feedstock to olefins, particularly light olefins, and showing system integration of a heavy olefins conversion zone in accordance with one embodiment.

FIG. 3 is a simplified schematic diagram of an integrated system for the processing of an oxygenate-containing feedstock to olefins, particularly light olefins, and showing system integration of a heavy olefins conversion zone in accordance with another embodiment.

FIG. 4 is a simplified schematic diagram of a RWD column or zone process modification in accordance with one preferred embodiment.

Those skilled in the art and guided by the teachings herein provided will recognize and appreciate that the illustrated system or process flow diagrams have been simplified by the elimination of various usual or customary pieces of process equipment including some heat exchangers, process control systems, pumps, fractionation systems, and the like. It may also be discerned that the process flow depicted in the figures may be modified in many aspects without departing from the basic overall concept of the invention.

### DETAILED DESCRIPTION OF THE INVENTION

Oxygenate-containing feedstock can be converted to light olefins in a catalytic reaction and heavier hydrocarbons (e.g., C₄+hydrocarbons) formed during such processing can be subsequently further processed to increase the light olefins (e.g., C₂ and C₃ olefins) produced or resulting therefrom. In accordance with a preferred embodiment, a methanol-containing feedstock is converted to form dimethyl ether (DME) which in turn is reacted to form a product mixture including light olefins and heavy olefins, with at least a portion of the heavy olefins being subsequently converted to form additional light olefin products.

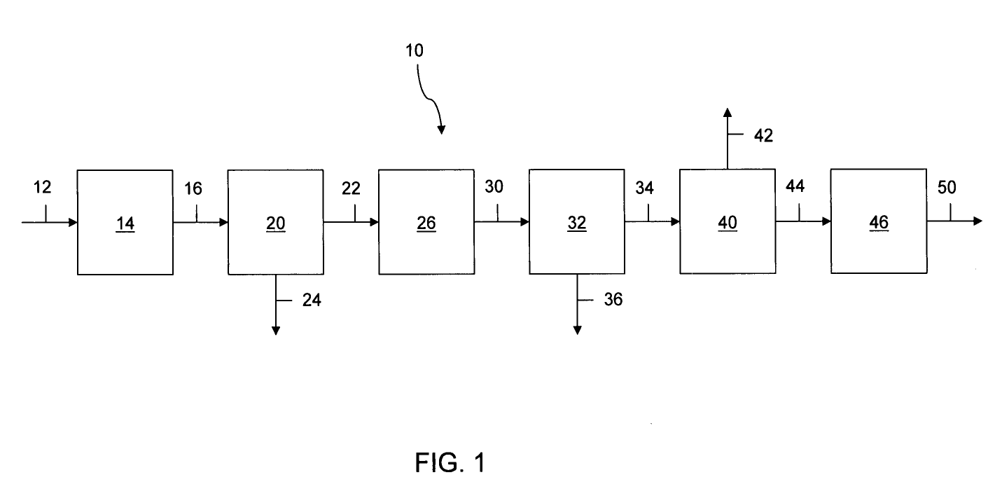

FIG. 1 schematically illustrates an integrated system, generally designated by the reference numeral 10, for processing of an oxygenate-containing feedstock to olefins, particularly light olefins, in accordance with one embodiment.

More particularly, a methanol-containing feedstock is introduced via a line 12 into a methanol conversion reactor zone 14 wherein the methanol-containing feedstock contacts with a methanol conversion catalyst and at reaction conditions effective to convert the methanol-containing feedstock to produce a methanol conversion reactor zone effluent stream comprising dimethyl ether and water, in a manner as is known in the art.

As will be appreciated by those skilled in the art and guided by the teachings herein provided, such a feedstock may be commercial grade methanol, crude methanol or any combination thereof. Crude methanol may be an unrefined product from a methanol synthesis unit. Those skilled in that art and guided by the teachings herein provided will understand and appreciate that in the interest of factors such as improved catalyst stability, embodiments utilizing higher purity methanol feeds may be preferred. Thus, suitable feeds may comprise methanol or a methanol and water blend, with possible such feeds having a methanol content of between about 65% and about 100% by weight, preferably a methanol content of between about 80% and about 100% by weight and, in accordance one preferred embodiment, a methanol content of between about 95% and about 100% by weight.

While the process conditions for such methanol conversion to dimethyl ether can vary, in practice such vapor phase process reaction can typically desirably occur at a temperature in the range of about 200° to about 300° C. (with a temperature of about 240° to about 260° C., e.g., at about 250° C., being preferred); a pressure in the range of about 200 to about 1500 kPa (with a pressure in the range of about 400 to about 700 kPa, e.g., at about 500 kPa, being preferred); and a weight hourly space velocity (“WHSV”) in the range of about 2 to about 15 hr⁻¹, with a WHSV in the range of about 3 to about 7 hr⁻¹, e.g., about 5 hr⁻¹, being preferred). In practice, a rate of conversion of methanol to dimethyl ether of about 80 percent or more is preferred.

The methanol conversion reactor zone effluent stream is introduced via a line 16 into a separator section 20 such as composed of one or more separation units such as known in the art wherein at least a portion of the water is removed therefrom to form a first process stream comprising dimethyl ether and having a reduced water content in a line 22 and a stream composed primarily of water, alone or in combination with unreacted methanol, in a line 24. As will be appreciated, a cooler device (not shown) may be appropriately disposed prior to the separator section 20 such as to facilitate desired water separation.

For example, such water separation can desirably be carried out in a flash drum or, if a more complete separation is desired, in a distillation column separation unit. In practice, it is generally desirable to remove at least about 75 percent or more, preferably at least about 90 percent or more of the produced water.

Those skilled in the art and guided by the teachings herein provided will appreciate that remaining unreacted methanol can either partition in a separation unit overhead stream or a separation unit bottoms stream or both, for further processing as herein described. For example, methanol in such separation unit bottoms stream can, if desired, be recovered (such as through or by a stripper column) and recycled to the methanol conversion reactor zone 14.

The first process stream or at least a portion thereof, is fed or introduced via the line 22 into an oxygenate conversion reactor section 26 wherein the feed contacts with an oxygenate conversion catalyst at reaction conditions effective to convert at least a portion of the feed to an oxygenate conversion product stream comprising fuel gas hydrocarbons, light olefins, and C₄+hydrocarbons, including a quantity of heavy hydrocarbons, in a manner as is known in the art, such as, for example, utilizing a fluidized bed reactor.

Reaction conditions for the conversion of oxygenates such as dimethyl ether, methanol and combinations thereof, for example, to light olefins are known to those skilled in the art. Preferably, in accordance with particular embodiments, reaction conditions comprise a temperature between about 200° and about 700° C., more preferably between about 300° and 600° C., and most preferably between about 400° and about 550° C. As will be appreciated by those skilled in the art and guided by the teachings herein provided, the reactions conditions are generally variable such as dependent on the desired products. The light olefins produced can have a ratio of ethylene to propylene of between about 0.5 and about 2.0 and preferably between about 0.75 and about 1.25. If a higher ratio of ethylene to propylene is desired, then the reaction temperature is higher than if a lower ratio of ethylene to propylene is desired. The preferred feed temperature range is between about 80° and about 210° C. More preferably the feed temperature range is between about 110° and 210° C. In accordance with one preferred embodiment, the temperature is desirably maintained below 210° C. to avoid or minimize thermal decomposition.

In accordance with certain preferred embodiments, it is particularly advantageous to employ oxygenate conversion reaction conditions including an oxygenate conversion reaction pressure of at least 240 kPa absolute. In certain preferred embodiments, an oxygenate conversion reaction pressure in a range of at least 240 kPa absolute to 580 kPa absolute is preferred. Moreover, in certain preferred embodiments an oxygenate conversion reaction pressure of at least 300 kPa absolute and such as in a range of at least 300 kPa absolute to 450 kPa absolute may be preferred. Those skilled in the art and guided by the teachings herein provided will appreciate that through such operation at pressures higher than normally utilized in conventional oxygenate-to-olefin, particularly methanol-to-olefin (e.g., “MTO”) processing, significant reductions in reactor size (e.g., reductions in size of the oxygenate conversion reactor can be realized). For example, in view of the ratio of pressure between normal operation and higher pressure operation in accordance herewith, reductions in reactor size of at least about 20 percent or more, such as reductions in reactor size of about 33 percent or more can be realized through such higher pressure operation.

In practice, oxygenate conversions of at least about 90 percent, preferably of at least about 95 percent and, in at least certain preferred embodiments, conversions of 98 to 99 percent or more can be realized in such oxygenate-to-olefin conversion processing.

The oxygenate conversion reactor section 26 produces or results in an oxygenate conversion product or effluent stream generally comprising fuel gas hydrocarbons, light olefins, heavy olefins and other C₄+hydrocarbons as well as by-product water in a line 30. The oxygenate conversion effluent stream or at least a portion thereof is appropriately processed such as through a quench and compressor section 32 such as to form a resulting compressed oxygenate conversion product stream in a line 34 and a wastewater stream in a line 36, such as, for example, may contain low levels of unreacted alcohols as well as small amounts of oxygenated byproducts such as low molecular weight aldehydes and organic acids, and such as may be appropriately treated and disposed or recycled.

The oxygenate conversion product stream line 34 is introduced into an appropriate gas concentration system 40.

Gas concentration systems such as used for the processing of the products resulting from such oxygenate conversion processing are well known to those skilled in the art and do not generally form limitations on the broader practice of the invention as those skilled in the art and guided by the teachings herein provided will appreciate.

In the gas concentration system 40, the oxygenate conversion product stream line 34, in whole or in part, is desirably processed to provide one or more desired process streams such as including one or more of a fuel gas stream, an ethylene stream, a propylene stream, a heavy olefins stream and a stream of other C₄+hydrocarbons. Those skilled in the art and guided by the teachings herein provided will appreciate that particular such process streams may desirably be utilized in specific embodiments as herein described below. FIG. 1 has been simplified to show a process stream line 42 such as generally composed of one or more end product materials and a process stream line 44 such as sent for further processing in accordance with the invention as more fully described below.

One or more of the process streams resulting from the gas concentration system 40 (in the FIG. 1 embodiment, the process stream in the line 44) is introduced into a heavy olefins conversion zone 46, such as more specifically described below, with at least a portion of such process stream appropriately reacted to form heavy olefins conversion zone effluent comprising at least additional light olefins, shown as exiting therefrom as a process stream line 50.

Those skilled in the art and guided by the teachings herein provided will appreciate that the system integration of the methanol conversion reactor zone whereby methanol can desirably be converted to dimethyl ether, with the subsequent removal of byproduct water reduces the volumetric flow through the reactor and hence reduces the size of the reactor. Moreover, such removal of water can advantageously reduce the hydrothermal severity of the reactor. Still further, the system integration of such a methanol conversion reactor zone can desirably result in removal of a significant portion of the heat of reaction such as to allow operation with reduced cooling requirements (e.g., operation with the removal of one or more catalyst coolers from the reactor). Yet still further, possible processing disadvantages such as due to possible increased selectivity to heavy hydrocarbons, particularly heavy olefins, are desirably minimized or avoided through the system integration of appropriate heavy olefins conversion zone as herein described.

Those skilled in the art and guided by the teachings herein provided will additionally note that the use of DME as feed to an oxygenate-to-olefins conversion reactor unit can present operational advantages over the use of other oxygenate feed materials, such as during the startup of the oxygenates-to-olefins reactor. For example, due to its relatively low boiling point, DME can be introduced as a gas into a cold reactor without the possibility of condensation, and can be used as a heating medium to increase the reactor temperature. In contrast, higher boiling oxygenate feedstock materials such as methanol, ethanol, etc, may require the reactor to be preheated such as by or through some other heating medium to avoid condensation in the reactor. Those skilled in the art will recognize and appreciate the importance of avoiding gas condensation in a fluidized bed system, and will recognize the advantages of a simplified startup procedure using DME as a feed material in such processing.

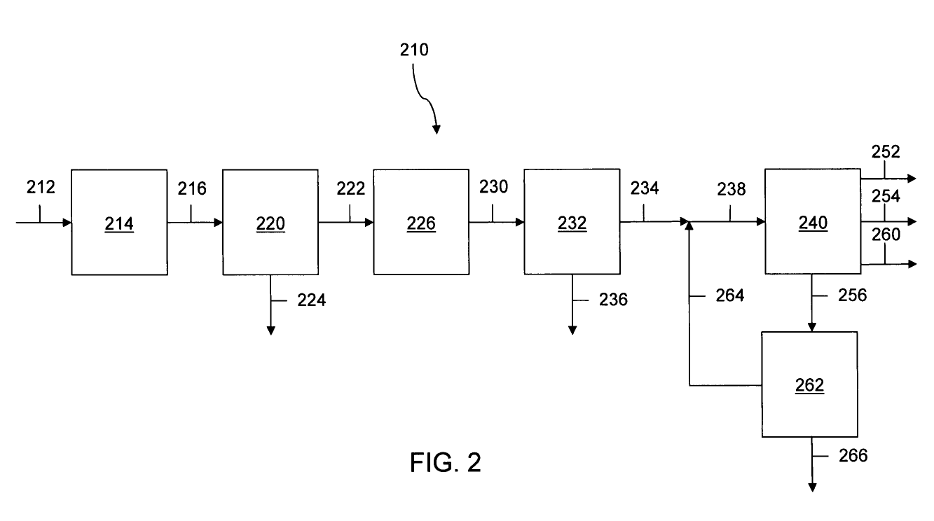

To further the understanding of the subject development, reference is now made to FIG. 2. FIG. 2 schematically illustrates an integrated system, generally designated by the reference numeral 210, for processing of an oxygenate-containing feedstock to olefins, particularly light olefins, and showing system integration of a heavy olefins conversion zone in accordance with one embodiment.

In the integrated system 210, similar to the integrated system 10 described above, a methanol-containing feedstock such as described above is introduced via a line 212 into a methanol conversion reactor zone 214 wherein the methanol-containing feedstock contacts with a methanol conversion catalyst and at reaction conditions effective to convert the methanol-containing feedstock to produce a methanol conversion reactor zone effluent stream such as comprising dimethyl ether and water.

The methanol conversion reactor zone effluent stream is introduced via a line 216 into a separator section 220 such as described above wherein water is removed therefrom to form a first process stream comprising dimethyl ether and having a reduced water content in a line 222 and a stream composed primarily of water, alone or in combination with unreacted methanol, in a line 224.

The first process stream, or at least a portion thereof, is fed or introduced via the line 222 into an oxygenate conversion reactor section 226 wherein the feed contacts with an oxygenate conversion catalyst at reaction conditions effective to convert at least a portion of the feed to an oxygenate conversion product stream comprising fuel gas hydrocarbons, light olefins, and C₄+hydrocarbons, including a quantity of heavy hydrocarbons, in a manner as is known in the art, such as, for example, utilizing a fluidized bed reactor, such as described above.

The oxygenate conversion reactor section 226 produces or results in an oxygenate conversion product or effluent stream generally comprising fuel gas hydrocarbons, light olefins, heavy olefins and other C₄+hydrocarbons as well as by-product water in a line 230. The oxygenate conversion effluent stream or at least a portion thereof is appropriately processed such as through a quench and compressor section 232 such as to form a resulting compressed oxygenate conversion product stream in a line 234 and a wastewater stream in a line 236, as described above.

The oxygenate conversion product stream can be passed, via the lines 234 and 238, and introduced into an appropriate gas concentration system 240. In the gas concentration system 240, the oxygenate conversion product stream, in whole or in part, is desirably processed as described above to provide one or more desired process streams such as including one or more of an ethylene stream such as in a line 252, a propylene stream in a line 254, a C₄+hydrocarbon stream, including C₄ and C₅ olefins, in a line 256 and one or more other process streams and such as may include a fuel gas stream, one or more paraffin purge streams, etc., and generally represented by the line 260.

The C₄+hydrocarbon stream or a selected portion thereof in the line 256 is introduced into an olefin cracking reactor section 262, such as in the form of a fixed bed reactor, as is known in the art and wherein such process stream materials contact with an olefin cracking catalyst and at reaction conditions, in a manner as is known in the art, effective to convert C₄ and C₅ olefins therein contained to a cracked olefins effluent stream comprising light olefins in a line 264.

A purge stream in a line 266 is shown whereby heavier materials such as C₄-C₆ paraffin compounds and the like may desirably be purged from the material stream being processed in the system 210, in a manner such as known in the art. As will be appreciated by those skilled in the art and guided by the teachings herein provided, such compounds generally do not convert very well in olefin cracking reactors. Consequently, such purging can avoid the undesirable build-up of such compounds within the system 210.

The cracked olefins effluent stream can be, as shown, desirably passed through the line 264 and the line 238 and appropriately processed through the gas concentration system 240.

As will be appreciated by those skilled in the art and guided by the teachings herein provided, such system integration of a heavy olefins conversion zone in the form of an olefin cracking reaction section can at least in part counteract increased selectivity to heavy hydrocarbons due to increased pressure operation.

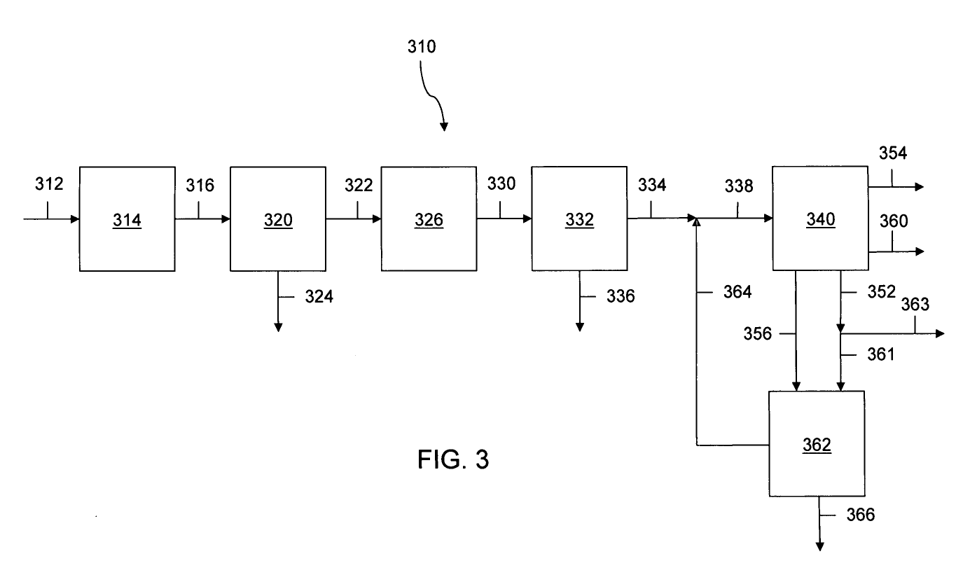

FIG. 3 schematically illustrates an integrated system, generally designated by the reference numeral 310, for processing of an oxygenate-containing feedstock to olefins, particularly light olefins, and showing system integration of a heavy olefins conversion zone in accordance with another embodiment.

In the integrated system 310, similar to the integrated system 10 described above, an appropriate methanol-containing feedstock such as described above is introduced via a line 312 into a methanol conversion reactor zone 314 wherein the methanol-containing feedstock contacts with a methanol conversion catalyst and at reaction conditions effective to convert the methanol-containing feedstock to produce a methanol conversion reactor zone effluent stream such as comprising dimethyl ether and water.

The methanol conversion reactor zone effluent stream is introduced via a line 316 into a separator section 320 such as described above wherein water is removed therefrom to form a first process stream comprising dimethyl ether and having a reduced water content in a line 322 and a stream composed primarily of water, alone or in combination with unreacted methanol, in a line 324.

The first process stream, or at least a portion thereof, is fed or introduced via the line 322 into an oxygenate conversion reactor section 326 wherein the feed contacts with an oxygenate conversion catalyst at reaction conditions effective to convert at least a portion of the feed to an oxygenate conversion product stream comprising fuel gas hydrocarbons, light olefins, and C₄+hydrocarbons, including a quantity of heavy hydrocarbons, in a manner as is known in the art, such as, for example, utilizing a fluidized bed reactor, such as described above.

The oxygenate conversion reactor section 326 produces or results in an oxygenate conversion product or effluent stream generally comprising fuel gas hydrocarbons, light olefins, heavy olefins and other C₄+hydrocarbons as well as by-product water in a line 330. The oxygenate conversion effluent stream or at least a portion thereof is appropriately processed such as through a quench and compressor section 332 such as to form a resulting compressed oxygenate conversion product stream in a line 334 and a wastewater stream in a line 336, as described above.

The oxygenate conversion product stream can be passed, via the lines 334 and 338, and introduced into an appropriate gas concentration system 340. In the gas concentration system 340, the oxygenate conversion product stream, in whole or in part, is desirably processed such as described above to provide one or more desired process streams such as including one or more of an ethylene stream such as in a line 352, a propylene stream in a line 354, a C₄ hydrocarbon stream, including C₄ olefins, in a line 356 and one or more other process streams and such as may include a fuel gas stream, one or more purge streams, etc., and generally represented by the line 360.

The C₄ hydrocarbon stream or a selected portion thereof in the line 356 and at least a portion of the ethylene stream in the line 352, such as shown by the line 361, are introduced into a heavy olefins conversion zone 362 in the form of a metathesis reaction section and under effective conditions to produce a metathesis effluent comprising propylene. The excess or net ethylene can be passed by the line 363 such as for product recovery or further processing as may be desired.

The metathesis reaction can generally be carried out under conditions and employs catalysts such as are known in the art. In accordance with one preferred embodiment, a metathesis catalyst such as containing a catalytic amount of at least one of molybdenum oxide and tungsten oxide is suitable for the metathesis reaction. Conditions for the metathesis reaction generally include reaction temperature ranging from about 20° to about 450° C., preferably 250° to 350° C., and pressures varying from about atmospheric to upwards of 3,000 psig (20.6 MPag), preferably between 435 and 510 psig (3000 to 3500 kpag), although higher pressures can be employed if desired. In general, the metathesis equilibrium for propylene production is generally favored by lower temperatures.

Catalysts which are active for the metathesis of olefins and which can be used in the process of this invention are of a generally known type. The disproportionation (metathesis) of butene with ethylene can, for example, be carried out in the vapor phase at about 300°-350° C. and about 0.5 MPa absolute (75 psia) with a WHSV of 50 to 100 and a once-through conversion of about 15% or more, depending on the ethylene to butene ratio.

Such metathesis catalysts may be homogeneous or heterogeneous, with heterogeneous catalysts being preferred. The metathesis catalyst preferably comprises a catalytically effective amount of transition metal component. The preferred transition metals for use in the present invention include tungsten, molybdenum, nickel, rhenium, and mixtures thereof. The transition metal component may be present as elemental metal and/or one or more compounds of the metal. If the catalyst is heterogeneous, it is preferred that the transition metal component be associated with a support. Any suitable support material may be employed provided that it does not substantially interfere with the feedstock components or the lower olefin component conversion. Preferably, the support material is an oxide, such as silica, alumina, titania, zirconia and mixtures thereof. Silica is a particularly preferred support material. If a support material is employed, the amount of transition metal component used in combination with the support material may vary widely depending, for example, on the particular application involved and/or the transition metal being used. Preferably, the transition metal comprises about 1% to about 20%, by weight (calculated as elemental metal) of the total catalyst. The metathesis catalyst advantageously comprises a catalytically effective amount of at least one of the above-noted transition metals capable of promoting olefin metathesis. The catalyst may also contain at least one activating agent present in an amount to improve the effectiveness of the catalyst. Various activating agents may be employed, including activating agents which are well known in the art to facilitate metathesis reactions. Light olefin metathesis catalysts can, for example, desirably be complexes of tungsten (W), molybdenum (Mo), or rhenium (Re) in a heterogeneous or homogeneous phase.

The metathesis effluent stream comprising propylene can be, as shown, desirably passed through a line 364 and the line 338 and appropriately processed through the gas concentration system 340.

A purge stream in a line 366 is shown whereby materials such as C₄ paraffin compounds and the like may desirably be purged from the system.

As will be appreciated by those skilled in the art and guided by the teachings herein provided, such system integration of a heavy olefins conversion zone in the form of a metathesis reaction section can at least in part counteract increased selectivity to heavy hydrocarbons, e.g., heavy olefins, due to increased pressure operation.

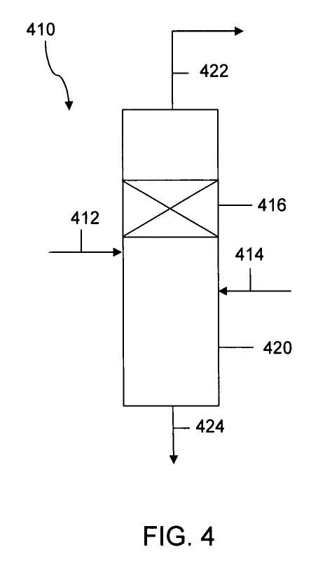

Turning now to FIG. 4, there is illustrated a simplified schematic diagram of a processing arrangement generally designated by the reference numeral 410 in accordance with one preferred embodiment.

More specifically, in the processing arrangement 410, a methanol-containing feedstock such as described above is introduced via a line 412 into a Reaction With Distillation (RWD) column or zone 414. The RWD column or zone desirably generally serves to combine reaction and distillation processing in a single processing apparatus. Thus, the RWD column or zone 414 can desirably serve to replace both the methanol conversion reactor zone 14 and the separator section 20 in the above described integrated system 10 shown in FIG. 1, for example.

U.S. Pat. No. 5,817,906 to Marker et al., the disclosure of which is hereby incorporated by reference in its entirety, discloses processing for producing light olefins using reaction with distillation processing.

The RWD zone 414 includes a reaction section 416 and a distillation section 420 such as wherein the methanol conversion catalyst is retained. As the methanol conversion occurs, a product effluent comprising dimethyl ether and having a reduced amount of water relative to the crude oxygenate feedstream is removed via a line 422 and concurrently water is produced and removed as a stream via a line 424.

With such processing, the energy provided by the heat of reaction of the methanol in the conversion over the acid catalyst can be advantageously employed to reboil the distillation section 420 to separate the ether product and unreacted methanol from the water stream which is removed from the bottom of the reaction with distillation zone 414. The reaction section 416 may be present at any point in the reaction with distillation zone 414. For the desired separation of ether product and unreacted methanol from water, it is generally preferred that the reaction section 416 be located at a point above the point where the methanol feedstock is introduced to the reaction with distillation zone 414. In this manner, excess water in the methanol feedstock can at least partially be removed in the distillation section 420 prior to entering the reaction section 416. This synergy provides a further advantage in reduced capital and utility costs for the invention over conventional processing schemes.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### EXAMPLES

In these simulation or model-based examples, a number of systems are considered for the conversion of a methanol feed, in a set amount, for the production of light olefins (ethylene and propylene), with emphasis on maximizing production of propylene.

### Comparative Example 1 (CE 1)

In this comparative example, the methanol feed is converted in an oxygenate-to-olefin fluidized bed reactor unit at a reaction pressure of 170 kPa and a low temperature suitable for maximum propylene selectivity. The reactor effluent is then fed to a separation system for purification of light olefins and rejection of by-products. Such separation systems are well known to those skilled in the art and typically include or are based on conventional methods of separation and purification, as would be found in a conventional plant for production of light olefins.

### Comparative Example 2 (CE 2)

In this comparative example, the methanol feed is converted in an oxygenate-to-olefin fluidized bed reactor unit at the elevated reaction pressure of 412 kPa and the same temperature as in Comparative Example 1. The resulting reactor effluent is then separated and purified to recover light olefins, as in Comparative Example 1.

### Comparative Example 3 (CE 3)

In this comparative example, the methanol feed is converted in a system that includes a methanol reaction zone for the conversion of methanol to DME and water, followed by a de-watering step in which 95% of the water is removed. A conversion of 85% is achieved in the methanol reaction zone. The resulting stream is then fed to an oxygenate-to-olefin fluidized bed reactor unit at the elevated reaction pressure of 412 kPa and the same temperature as in Comparative Example 1. The resulting reactor effluent is then separated and purified to recover light olefins, as in Comparative Example 1.

### Comparative Example 4 (CE 4)

In this comparative example, the methanol feed is converted in an oxygenate-to-olefin fluidized bed reactor unit at the elevated reaction pressure of 412 kPa (as in Comparative Example 2) and the same temperature as in Comparative Example 1. The resulting reactor effluent is then separated and purified to recover light olefins, as in Comparative Example 1. In this comparative example, however, the heavy olefin by-products, primarily composed of butene, pentene, and hexene, are fed to a heavy olefin conversion zone. The effluent from the heavy olefin conversion zone is then returned to the separation system for the recovery of light olefins therefrom. A purge of heavy material results from the heavy olefin conversion zone.

### Example 1

In this example, an integrated system consistent with the subject development is used. More specifically, the methanol feed is converted in a system that includes a methanol reaction zone for the conversion of methanol to DME and water, followed by a de-watering step in which 95% of the water is removed. A conversion of 85% is achieved in the methanol reaction zone. The resulting stream is then fed to an oxygenate-to-olefin fluidized bed reactor unit at the elevated reaction pressure of 412 kPa and the same temperature as in the comparative examples. The resulting reactor effluent is then separated and purified to recover light olefins, as in Comparative Example 1. The heavy olefin by-products, primarily composed of butene, pentene, and hexene, are fed to a heavy olefin conversion zone. The effluent from the heavy olefin conversion zone is then returned to the separation system for the recovery of light olefins therefrom. A purge of heavy material results from the heavy olefin conversion zone.

### Results

For each of these examples, the propylene yield (defined as the weight percentage of carbon atoms contained in the feed which are converted to propylene) is calculated using a yield simulation model and shown in the TABLE, below. Also, for each of these examples, the volumetric flowrate (defined as the actual volumetric flow relative to the volumetric flow rate in Comparative Example 1) is determined using a process simulation model and is also shown in the TABLE, below.

TABLE

| Example | Propylene Yield (%) | Relative Volumetric Flow Rate (%) |
|---|---|---|
| CE 1 | 43.9 | 100 |
| CE 2 | 44.6 | 63 |
| CE 3 | 44.9 | 42 |
| CE 4 | 57.0 | 63 |
| Example 1 | 58.1 | 42 |

### Discussion of Results

As shown in the TABLE, the integrated system of Example 1 achieves a higher propylene yield than any of the comparative examples. As further shown in the TABLE, the integrated system of Example 1 also simultaneously permits a significant reduction in the volumetric flowrate through the reactor. A person skilled in the art and guided by the teachings herein provided will appreciate and recognize that as a fluidized reactor system typically comprises a major cost component of an operating plant, significant reductions in reactor size and corresponding savings in reactor and catalyst inventory costs associated therewith can be realized through the practice of the invention.

The invention thus provides processing schemes and arrangements for the production of olefins and, more particularly, for the production of light olefins from an oxygenate-containing feed and which processing schemes and arrangements are advantageously simpler, more effective and/or more efficient than heretofore been generally available.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A method for producing light olefins, said method comprising:
contacting a methanol-containing feedstock in a methanol conversion reactor zone with a catalyst and at reaction conditions effective to produce a methanol conversion reactor zone effluent comprising dimethyl ether and water;
removing at least a portion of the water from the methanol conversion reactor zone effluent to form a first process stream comprising dimethyl ether and having a reduced water content;
contacting a feed comprising at least a portion of the first process stream in an oxygenate conversion reactor zone with an oxygenate conversion catalyst at oxygenate conversion reaction conditions effective to convert at least a portion of the feed to an oxygenate conversion product stream comprising light olefins and heavy olefins, wherein the oxygenate conversion reaction conditions include an oxygenate conversion reaction pressure of at least 240 kPa absolute;
reacting at least a portion of the oxygenate conversion product stream heavy olefins in a heavy olefins conversion zone to form a heavy olefins conversion zone effluent stream comprising additional light olefins; and
recovering at least a portion of the additional light olefins from the heavy olefins conversion zone effluent stream.

2. The method of claim 1 wherein the oxygenate conversion reaction pressure is in a range of at least 240 kPa absolute to 580 kPa absolute.

3. The method of claim 1 wherein the oxygenate conversion reaction pressure is at least 300 kPa absolute.

4. The method of claim 3 wherein the oxygenate conversion reaction pressure is in a range of at least 300 kPa absolute to 450 kPa absolute.

5. The method of claim 1 wherein the reaction of at least a portion of the oxygenate conversion product stream heavy olefins comprises at least one of an olefin cracking reaction and a metathesis reaction.

6. The method of claim 5 wherein, prior to the reaction of at least a portion of the oxygenate conversion product stream heavy olefins, the method additionally comprises at least partially separating the light olefins from the heavy olefins of the oxygenate conversion product stream.

7. The method of claim 6 wherein the reaction of at least a portion of the oxygenate conversion product stream heavy olefins comprises cracking at least a portion of the separated heavy olefins to form a cracked olefin effluent comprising C₂ and C₃ olefins.

8. The method of claim 5 wherein the light olefins of the oxygenate conversion product stream comprise a quantity of C₂ olefins and the heavy olefins of the oxygenate conversion product stream comprise a quantity of C₄ olefins and wherein the reaction of at least a portion of the oxygenate conversion product stream heavy olefins comprises contacting at least a portion of the C₄ olefins with at least a portion of the C₂ olefins in a metathesis section at effective conditions to produce a metathesis effluent comprising C₃ olefins.

9. The method of claim 8 wherein C₂ and C₄ olefins are introduced into the metathesis section in a molar ratio of about 2 to about 3 moles of C₂ olefins per mole of C₄ olefins.

10. The method of claim 1 wherein the contacting of the methanol-containing feedstock in the methanol conversion reactor zone with a catalyst and at reaction conditions effective to produce a methanol conversion reactor zone effluent comprising dimethyl ether and water and the removing of at least a portion of the water from the methanol conversion reactor zone effluent to form a first process stream comprising dimethyl ether and having a reduced water content occurs concurrently in a single reaction with distillation zone.

11. A method for producing light olefins, said method comprising:
contacting a methanol-containing feedstock in a methanol conversion reactor zone with a catalyst and at reaction conditions effective to produce a methanol conversion reactor zone effluent comprising dimethyl ether and water;
removing at least a portion of the water from the methanol conversion reactor zone effluent to form a first process stream comprising dimethyl ether and having a reduced water content;
contacting a feed comprising at least a portion of the first process stream in an oxygenate conversion reactor zone with an oxygenate conversion catalyst at oxygenate conversion reaction conditions effective to convert at least a portion of the feed to an oxygenate conversion product stream comprising light olefins and heavy olefins, wherein the oxygenate conversion reaction conditions include an oxygenate conversion reaction pressure in a range of at least 300 kPa absolute to 450 kPa absolute;
reacting at least a portion of the oxygenate conversion product stream heavy olefins in a heavy olefins conversion zone via at least one of an olefin cracking reaction and a metathesis reaction to form a heavy olefins conversion zone effluent stream comprising additional light olefins; and
recovering at least a portion of the additional light olefins from the heavy olefins conversion zone effluent stream.

12. The method of claim 11 wherein the reaction of at least a portion of the oxygenate conversion product stream heavy olefins comprises cracking at least a portion of the separated heavy olefins to form a cracked olefin effluent comprising C₂ and C₃ olefins.

13. The method of claim 11 wherein the light olefins of the oxygenate conversion product stream comprise a quantity of C₂ olefins and the heavy olefins of the oxygenate conversion product stream comprise a quantity of C₄ olefins and wherein the reaction of at least a portion of the oxygenate conversion product stream heavy olefins comprises contacting at least a portion of the C₄ olefins with at least a portion of the C₂ olefins in a metathesis section at effective conditions to produce a metathesis effluent comprising C₃ olefins.

14. The method of claim 13 wherein C₂ and C₄ olefins are introduced into the metathesis section in a molar ratio of about 2 to about 3 moles of C₂ olefins per mole of C₄ olefins.

15. The method of claim 11 wherein the contacting of the methanol-containing feedstock in the methanol conversion reactor zone with a catalyst and at reaction conditions effective to produce a methanol conversion reactor zone effluent comprising dimethyl ether and water and the removing of at least a portion of the water from the methanol conversion reactor zone effluent to form a first process stream comprising dimethyl ether and having a reduced water content occurs concurrently in a single reaction with distillation zone.

16. A system for producing light olefins, said system comprising:
a methanol conversion reactor zone for contacting a methanol-containing feedstock with a catalyst and at reaction conditions effective to produce a methanol conversion reactor zone effluent comprising dimethyl ether and water;
a first separator effective to separate at least a portion of the water from the methanol conversion reactor zone effluent to form a first process stream comprising dimethyl ether and having a reduced water content;
an oxygenate conversion reactor zone for contacting a feed comprising at least a portion of the first process stream dimethyl ether with an oxygenate conversion with a catalyst and at reaction conditions including a reaction pressure of at least 240 kPa absolute effective to convert at least a portion of the feed to an oxygenate conversion product stream comprising light olefins and heavy olefins;
a heavy olefins conversion zone effective to convert oxygenate conversion product stream heavy olefins to form a heavy olefins conversion zone effluent stream comprising additional light olefins; and
a recovery zone for recovering at least a portion of the additional light olefins from the heavy olefins conversion zone effluent stream.

17. The system of claim 16 wherein the methanol conversion reactor zone and the first separator are at least in part combined in the form of a RWD column.

18. The system of claim 16 additionally comprising a second separator effective to at least partially separate the light olefins from the heavy olefins of the oxygenate conversion product stream.

19. The system of claim 18 wherein the heavy olefins conversion zone comprises an olefin cracking reactor section to crack at least a portion of the separated heavy olefins to form a cracked olefin effluent comprising C₂ and C₃ olefins.

20. The system of claim 16 wherein the light olefins of the oxygenate conversion product stream comprise a quantity of C₂ olefins and the heavy olefins of the oxygenate conversion product stream comprise a quantity of C₄ olefins and wherein the heavy olefins conversion zone comprises a metathesis section wherein at least a portion of the C₄ olefins metathesize with at least a portion of the C₂ olefins to produce a metathesis effluent comprising C₃ olefins.
